(19) United States
(12) Patent Application Publication
(10) Pub. No.: US 2021/0032020 A1
(43) Pub. Date: Feb. 4, 2021

(54) SECURE DISPOSAL BAGS

(71) Applicant: LOOPEEZE LIMITED, London (GB)

(72) Inventor: Martha Silcott, London (GB)

(73) Assignee: LOOPEEZE LIMITED, London (GB)

(21) Appl. No.: 16/964,271

(22) PCT Filed: Feb. 5, 2019
(86) PCT No.: PCT/EP2019/052816

§ 371 Date: Jul. 23, 2020

(30) Foreign Application Priority Data
Feb. 5, 2018 (GB) 1801853.1
Jul. 30, 2018 (GB) 1812414.9

(51) Int. Cl. B65F 1/00; B65D 33/00; B65D 33/16; B65D 33/18

(52) U.S. Cl. CPC B65F 1/0006; B65D 33/18; B65D 33/1658; B65D 33/007

(57) ABSTRACT

A disposal bag (1) is disclosed of generally rectangular two-layer construction, with the layers sealed together at their edges on three sides of the rectangle (2, 3, 4). The side walls of the bag are overlaid on the openable side of the bag with extensions (5) which taper inwardly from the edges of the bag and extend outwardly to overlie the outer walls of the bag. The narrower end of each extension (5) is securely fastened to the exterior side wall of the bag at a position (6) so as to form a tunnel (7) each side of the bag into which the thumb and at least one finger may be inserted. At least one of the inside walls of the bag, at the open end of the bag or slightly inset from the open end of the bag, carries a continuous stripe of adhesive material, overlaid by a peel-off strip (9) of release-coated material. The adhesive material is of such a nature that once the peel-off strip (9) has been removed and the sides of the bag are brought into contact with one another, they bond together immediately to seal the disposal bag. The bag is of particular use for disposal of ostomy bags, tampons, sanitary or incontinence towels, catheters and condoms.

This invention relates to secure disposal bags, in particular for the disposal of contaminated items.

It is common practice to dispose of waste materials by placing them in a bag. Where the waste material is toxic, contaminated or merely unpleasant, it is desirable that, following the placement of the material into the bag, it is then sealed.

Particular problems arise in the case of items which need to be disposed of which may be contaminated with body fluids, for example tampons, sanitary or incontinence towels, diapers, condoms or catheters, or which may contain faecal material, for example ostomy bags. Desirably, these should be able to be placed simply and quickly into a disposal bag without transferring contamination to the hands of the user or to the exterior of the bag itself, and thereafter the disposal bag itself should be able to be securely sealed in a simple fashion not needing any complex manipulation. A known method of sealing is to provide an adhesive strip on part of the bag which can be covered with a protective strip of release paper until the user wishes to seal the bag.

U.S. Pat. No. 2,766,927 discloses a plastics bag construction for the receipt of a sanitary pad. The open end of the bag is folded out and down to form a cuff enabling the bag to be opened by inserting the fingers and thumb on opposite sides of the bag and moving them apart. The cuff can be unfolded once the sanitary pad has been placed in the bag and the bag sealed by bringing two adhesive strips, exposed by unfolding the cuff, together.

Patent Specification JP 2007253977A discloses a short cuff extending across the mouth of an openable bag which enables the user (by using the fingers of two hands) to pull the sides of the bag apart against the closure force of a sealing bead running across the interior of the bag.

WO 2007/077172 discloses a number of constructions of disposal bags configured as pouches, particularly ones suited for the disposal of used tampons, sanitary or incontinence towels. As shown for example in FIGS. 8 to 13, 16 and 17 of that specification, a disposal bag may be configured as a generally rectangular pouch sealed on three sides, and openable on an unsealed short side of the rectangle. Opening is facilitated by a pair of loops either side of the opening, sized to enable the thumb and a finger of the user to enable the sides to be moved apart when the thumb and finger are moved apart.

Such disposal bags can thus be easily opened using the finger and thumb of one hand inserted into the loops, enabling the user then to insert a used tampon or other article and then close the bag after having removed a peel-away protective strip covering a strip of adhesive running across the open top of the bag. When the bag is closed, the adhesive holds the two sides of the bag together.

The use of loops of material to assist in opening a mouth at the end of a medical fluid container is disclosed in US 2013/0066292 A. Specification WO2012/066865 discloses a loop on the exterior of a flexible container for a medical liquid into which the fingers may be inserted to assist in holding the container while opening its mouth by squeezing from the sides.

We have now found that by adopting a particular design for the openable end of a disposal bag, a secure disposal bag may be provided which can be safely used with contaminated items such as ostomy bags, tampons, sanitary or incontinence towels, and condoms to provide an easily operable disposal bag. The design avoids the risk of contamination from the item in turn contaminating the hand of the user, while retaining the feature that once used, the bag forms a secure sealed unit surrounding the contaminated items or material placed therein.

In accordance with the present invention, there is provided a disposal bag of generally rectangular two-layer construction, with the layers sealed together at their edges on two or three sides of the rectangle and having means on the outside of the side walls of the bag to enable them to be parted to form a mouth for the bag, and wherein at least one of the inside walls of the bag, at the open end of the bag or slightly inset from the open end of the bag, carries a continuous stripe of adhesive material, overlaid by a peel-off strip of release-coated material, the adhesive being of such a nature that once the peel-off strip has been removed and the sides of the bag are brought into contact with one another, they immediately become bonded together to close the disposal bag and characterised in that the means enabling the mouth of the bag to be opened comprise an outer cover sheet on each of the two side walls of the bag and each extending from a wider end attached to each side wall adjacent the openable side of the bag and extending across substantially the entire width of the bag, and each cover sheet tapering inwardly from the edges of the bag to a narrower end securely fastened to the exterior side wall of the bag at a position so as to form a tunnel each side of the bag of size sufficient to enable the thumb and at least one finger of the user to be inserted and moved apart to open the mouth of the bag.

Preferably the outer cover sheets are formed integrally with the sides of the bag, each side wall of the bag being extended on the openable side of the bag by an extension tapering at least initially inwardly from the edges of the bag and being turned outwardly from the mouth of the bag to overlie the outer walls of the bag, with the far end of each extension being securely fastened to the exterior side wall of the bag.

The adhesive material is preferably of such a nature that once the peel-off strip has been removed and the sides of the bag are brought into contact with one another, they bond together immediately to close the disposal bag with the bond strength of the adhesive bond so formed being greater than that of the tear strength of the material of the bag itself.

The provision of the wide tabs, the wider end of which extends across substantially the entire width of the mouth of the bag, enables the bag to be opened fully prior to the placing of the item(s) to be disposed of in the bag, even though the user's finger and thumb extend only part way across the bag, conveniently about half way. Insertion of the finger and thumb causes the material of the bag to form a sort of cylindrical or tubular channel which is not very bendy, and which acts, as the finger and thumb are moved apart, to open the mouth of the bag wide.

This and the dimensioning of the tunnel-forming portions and the design of the tunnels so that only partial insertion of the user's digits enables the bag to be opened entirely, facilitates the insertion into the bag of the item to be disposed of.

The bag is preferably made of plastics film material, though other flexible fabric or paper-like sheet materials may be used. For example low, medium or high density polyethylene film, or a biodegradable polymer film may be selected, of thickness sufficient to provide adequate strength while avoiding too great stiffness. Treated papers of adequate strength and impermeability may also be used, and if a coated paper is used the coating is preferably biodegradable. The film may be plain or coated, e.g. metallised. A film thickness of 50 to 90 microns for plastics film usually provides adequate strength without requiring excess material usage. The ends of the extensions or the loose outer cover sheets are preferably heat sealed to the external surface of the bag each side. Adhesive may be used in place of heat sealing, but heat sealing is preferred as it is less consumptive of raw material. The material of the bag is preferably opaque, and may also have odour reducing properties or be coated with a deodorant material to ensure that the bag once sealed is odour-free.

The far ends of each tapering extending portion of each side or of the outer cover sheets are preferably cut transversely, with the width of the transverse cut being at least 50% of the width of the bag and preferably 65% to 75% of the width of the bag. The transverse cut ends or the far ends of the outer cover sheets are preferably sealed or adhered to the bag exterior at a position spaced from the openable end of the bag by a sufficient distance to enable the material to form the tunnels on each side of the bag. The tunnel configuration promotes total opening of the end of the bag across its entire width, even though the finger(s) and thumb extend only part way across the bag. For ostomy bag disposal bags, the position of the narrower end of the cover sheet is preferably about a third of the way down the bag. For smaller disposal bags, for example for tampons, sanitary or incontinence towels, or condoms, it may be about half way between the openable and closed ends of the bag.

The taper on the extending portions of each side of the bag or on the outer cover sheets may be a straight line taper, but it is preferred that the taper angle is increased adjacent the open end of the bag and decreases towards the usually transversely cut end of the portion overlying each side of the bag. The degree of taper may even decrease to zero, with the far end then widening out a little. This curved tapering provides tunnels on each side of the bag with a concave arched entry opening into which it is easier to insert the finger(s) and thumb of one hand so that when the thumb and finger(s) are parted, the top of the bag opens easily to provide a wide aperture enabling the material or items to be disposed of to be easily inserted (or if the bag serves as packaging, to enable an unused item to be extracted). Following the insertion of an item for disposal, the release strip or strips is/are peeled off, and the bag then sealed completely by pressing the two sides together so that the adhesive instantly and permanently bonds the previously open end of the bag to form a completely sealed unit. This prevents any leakage and has the additional advantage of odour prevention. The thumb and finger(s) may be removed from the tunnels on opposite sides of the bag before sealing, or used to close the mouth of the bag by pressing the sides of the bag together,

The dimensions of the bag may vary within reasonable limits, and may be chosen, for example, to provide a bag adapted in terms of its size for disposal of a particular product, for example an ostomy bag, incontinence pad, or a condom.

The corners of the generally rectangular bag may also be rounded to better distribute the stresses at the edges of the bag once filled and sealed.

By way of example, disposal bags according to the present invention are illustrated in the accompanying diagrammatic drawings in which:

FIG. 1 is a diagrammatic side view of a disposal bag in accordance with the present invention suitable for the disposal of ostomy bags, in its ready-for-use condition; and

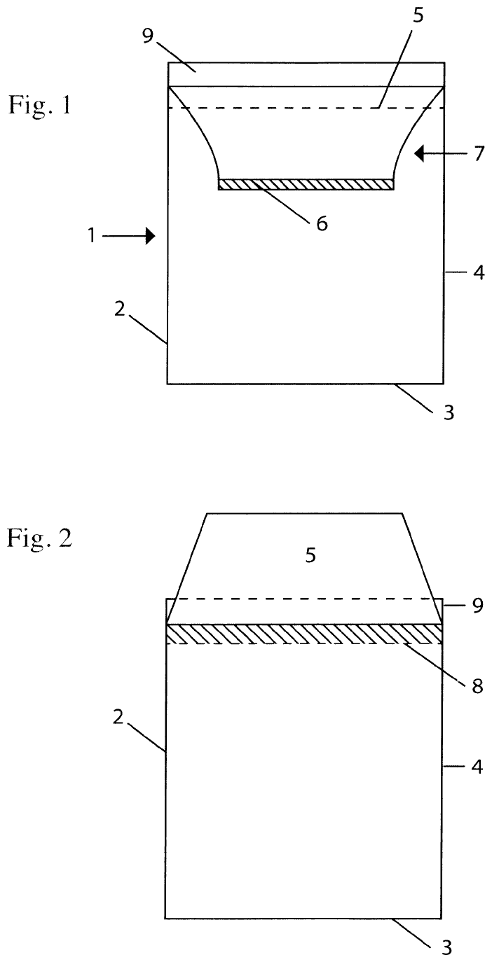

FIG. 2 shows the bag of FIG. 1 before the final manufacturing step of folding the projecting extensions outwardly and downwardly and fixing them to the exterior surface of the bag;

FIG. 3 shows the bag of FIG. 1 opened to receive an ostomy bag;

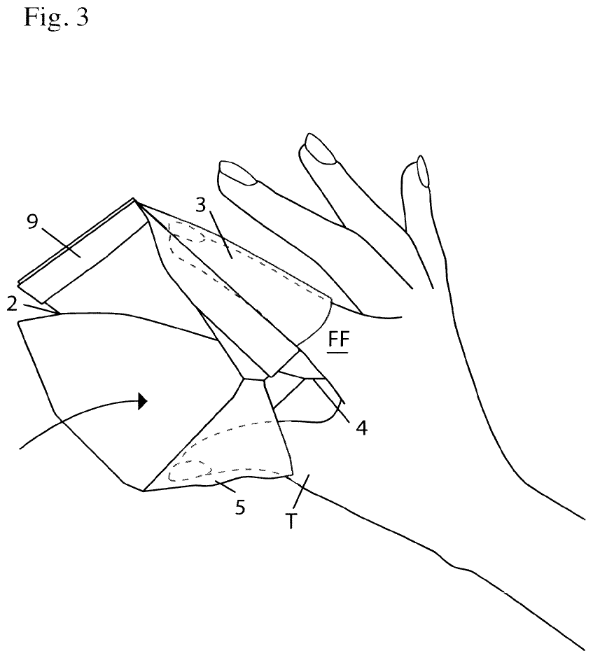

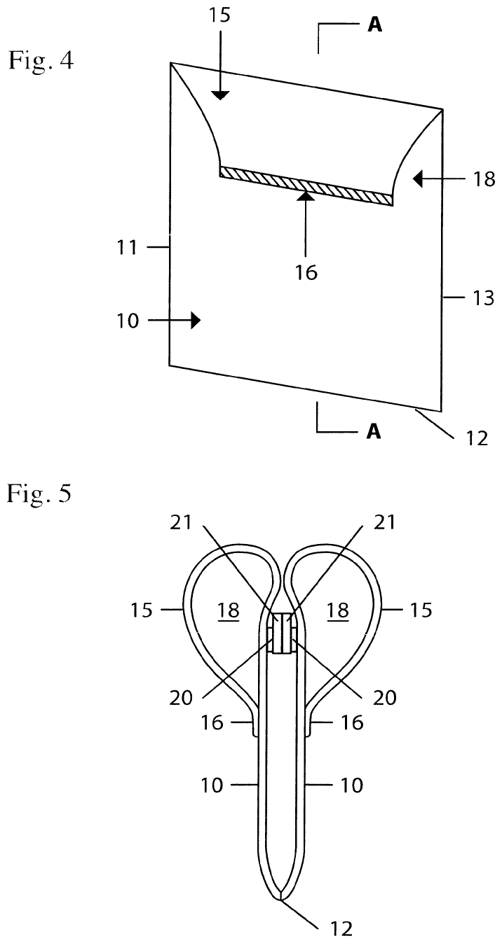

FIG. 4 is a diagrammatic view of a condom disposal bag according to the invention;

FIG. 5 is a diagrammatic section through the condom disposal bag of FIG. 4 along the section lines A-A; and

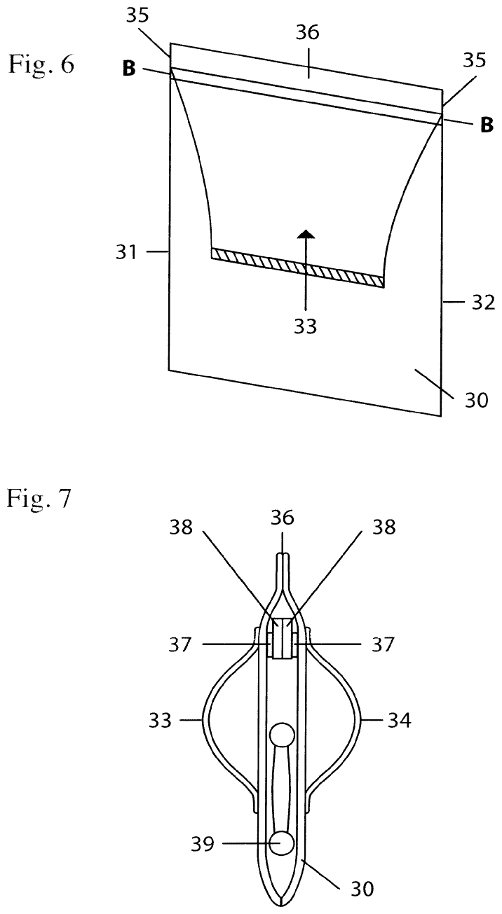

FIGS. 6 and 7 are respectively a diagrammatic view and section of an alternative form of condom package which incorporates a condom disposal bag according to the present invention.

Referring to FIGS. 1 to 3 of the drawings, a two-layer bag 1 is shown of rectangular shape, with welded edges 2, 3 and 4. The material of the bag extends in two generally trapezoidal flap portions 5 which in the finished bag have their narrower ends welded or adhered on to the sides of the bag as shown by hatched area 6 in FIG. 1. If desired, edges 2 and 4 may be welded and edge 3 formed by a fold.

Because of the natural resilience of the material from which the bag is made, and the positioning of the narrower end of each flap on the side of the bag, the folded-down flap portions adopt near the top of the bag, position slightly away from the generally flat rectangular shape of the bag to form the entrances to two tunnels into which the thumb or one or two fingers can be inserted in the direction of arrow 7.

On one (or both) the interior surfaces of the bag, near the openable end, is a stripe of adhesive material, located where shown by the hatched portion 8 in FIG. 2, covered by a strip of material 9 such as release-coated paper or made of a plastics film which can be easily and cleanly peeled off the adhesive stripe. Strip(s) 9 is/are wider than the adhesive stripe and project(s) above the top of the finished bag, as shown in FIG. 1, enabling it to be easily grasped and peeled away when the bag is to be sealed.

A convenient size for ostomy bag disposal bags as shown in FIGS. 1 and 2 is within the ranges of 130 to 170 mm wide and 180 to 220 mm long.

FIG. 3 shows the bag opened and ready for the receipt of an ostomy bag. As can be seen in this figure, the thumb T and first finger FF of a user's right hand have been inserted into the tunnels formed by flap portions 5 and then moved apart, so opening the mouth of the bag wide, even though the thumb T and first finger FF extend only part way across the bag. Insertion of the thumb and first finger forms the material of the bag into a cylinder or tube which acts as a relatively unbendable channel which enables the bag to be opened wide enough to enable easy insertion of the item to be disposed of as shown by the arrow on the left in FIG. 3.

Turning now to FIGS. 4 and 5, these show a condom disposal bag in accordance with the present invention. FIG. 4 is a side view of the bag from one side while FIG. 5 is a diagrammatic section through FIG. 4 along the lines A-A but with the thickness (but not the other dimensions) of the components enlarged for clarity.

Referring to FIGS. 4 and 5, the condom disposal bag shown is formed of two sheets of plastics material 10 which are welded together at three edges 11, 12, 13.

Each of the sides 10 has a tapered extension 15 which, at its narrow transversely-cut end 16, is heat-welded to the exterior of side 10.

As can be seen, by appropriate positioning of the end 16 and appropriate choice of the material from which the bag is made, the two tapered extensions 15 form tunnels 18.

Running between edges 11 and 13 across the bag are two strips of adhesive 20. The strips 20 are overlaid by strips of release paper 21. The pieces of release paper 21 are adhered to the respective adhesive strips 20, but not to one another. Just above the top of each release strip 21, the two extensions 15 are lightly adhered together at 23. This may be done with adhesive but is preferably done by carefully controlled heat sealing the two films together.

As shown in FIGS. 4 and 5, the disposal bag is empty. However, it may serve as a container for a conventionally packaged condom, or as a container for the unused condom itself.

In the latter case, when someone desires to use the condom, the finger(s) and thumb of the user are inserted into the tunnels 18, and then moved apart, whereupon the bag opens with rupture of the weak adhesive connection 23. The condom may then be extracted from the bag, any further standard packaging removed, and the condom then used for its customary purpose.

Following such use, the condom may be deposited back in the disposal bag by opening its top and dropping the condom in. Because the two strips of release paper are still in place, the ability of the disposal bag to be adhesively sealed is not impaired. If the bag had an adhesive strip 20 and release strip 21 only on one side, there would be a risk that the inside wall of the bag could become contaminated, particularly with lubricant associated with condom use, whereafter the adhesive bond to such a contaminated inner surface might not be effective. By using two adhesive strips, this problem is avoided.

Once the condom has been deposited into the disposal bag, the two release strips 21 are peeled off the adhesive strips 20 and the bag then closed by bringing the finger(s) and thumb together. The adhesive strips 20 adhere firmly to one another and, because they extend all the way across the bag, the disposal bag is then sealed.

FIGS. 6 and 7 show an alternative condom packaging/disposal bag structure. In this embodiment, the condom packaging consists of a folded strip 30, for example of a plastics film with welded edges 31, 32 and a welded top 33. The edges of the strip 30 are partly zigzag cut at 35 to enable the condom package to be opened by tearing the top portion off. Welded to the sides of the folded foil strip are two tapered cover sheets 33, 34. The weld between cover sheets 33, 34 and strip 30 nearer the top of the package acts to define the edge of the tear if the top portion is torn off right to left as shown in FIG. 6, along the line B-B. The tear may be assisted by part-perforation of the strip 30 along the line B-B if desired.

FIG. 7 shows the section of the package before use. As in FIG. 5, the thicknesses of the foil and sheet materials, and of the adhesive strips and release papers are enlarged to provide clarity. The adhesive strips are denoted 37 and the release papers 38. The condom is denoted 39.

When the package shown in FIGS. 6 and 7 is to be used, the top is first torn off and the fingers or finger and thumb inserted into the tunnels formed by cover sheets 33 and 34. By pulling the sides apart the condom can then be extracted and used. Once used and needing to be disposed of, the mouth of the package is then opened and the used condom placed in it. The release foil 38 is then pulled off, and the sides of the package moved together to adhere the two adhesive strips 37 together and seal the package.

In place of the two strips of release paper 38 as illustrated, there may be an extended strip of release paper, with each end adhered to the adhesive strip and with an intermediate section between them having three transverse folds in it, two valley folds adjacent the ends of the adhesive strips and a peak fold between them which can be easily grasped when the disposal bag is held open just after the used condom has been inserted.

1. A disposal bag of generally rectangular two-layer construction, with the layers sealed together at their edges on two or three sides of the rectangle and having means on the outside of the side walls of the bag to enable them to be parted to form a mouth for the bag, and wherein at least one of the inside walls of the bag, at the open end of the bag or slightly inset from the open end of the bag, carries a continuous stripe of adhesive material, overlaid by a peel-off strip of release-coated material, the adhesive being of such a nature that once the peel-off strip has been removed and the sides of the bag are brought into contact with one another, they immediately become bonded together to close the disposal bag and characterised in that the means enabling the mouth of the bag to be opened comprise an outer cover sheet on each of the two side walls of the bag and each extending from a wider end attached to each side wall adjacent the openable side of the bag and extending across substantially the entire width of the bag, and each cover sheet tapering inwardly from the edges of the bag to a narrower end securely fastened to the exterior side wall of the bag at a position so as to form a tunnel each side of the bag of size sufficient to enable the thumb and at least one finger of the user to be inserted and moved apart to open the mouth of the bag.

2. The disposal bag according to claim 1 wherein the outer cover sheets are formed integrally with the sides of the bag, each side wall of the bag being extended on the openable side of the bag by an extension tapering at least initially inwardly from the edges of the bag and being turned outwardly from the mouth of the bag to overlie the outer walls of the bag, with the far end of each extension being securely fastened to the exterior side wall of the bag.

3. The disposal bag according to claim 1 wherein the adhesive material is of such a nature that once the peel-off strip has been removed and the sides of the bag are brought into contact with one another, they bond together immediately to close the disposal bag with the bond strength of the adhesive bond so formed being greater than that of the tear strength of the material of the bag itself.

4. The disposal bag according to claim 1 wherein the material of the bag is a plastics film material of film thickness of 50 to 90 microns.

5. The disposal bag according to claim 1 wherein the ends of the extensions or of the tapered cover sheets are heat sealed to the external surface of the bag each side.

6. The disposal bag according to claim 1 wherein the material of the bag is opaque.

7. The disposal bag according to claim 1 wherein the narrower ends of each tapering extending portion of each side or of the cover sheets are cut transversely, with the width of the transverse cut being at least 50% of the width of the bag.

8. The disposal bag according to claim 7 wherein the width of the transverse cut is 65% to 75% of the width of the bag.

9. The disposal bag according to claim 1 wherein the taper angle of the outer cover sheets is increased adjacent the mouth of the bag and decreases away from the mouth.
